(19) United States
(12) Patent Application Publication
(10) Pub. No.: US 2017/0189557 A1
(43) Pub. Date: Jul. 6, 2017

(54) METHOD OF PREPARING FLUORESCENT SILK PROTEIN SOLUTION EXTRACTED FROM TRANSGENIC SILKWORM COCOONS AND METHOD OF MANUFACTURING SUPPORT USING THE SAME

(71) Applicant: Industry Academic Cooperation Foundation, Hallym University, Chuncheon-Si (KR)

(72) Inventors: Chan Hum Park, Chuncheon-Si (KR); Soo Hyeon Kim, Namyangju-Si (KR); Dong Wook Kim, Seoul (KR); Bo Mi Moon, Seoul (KR); Hyun Jung Park, Incheon (KR); Ye Ri Park, Chuncheon-Si (KR); Min Chae Lee, Guri-Si (KR); Ok Joo Lee, Jinbu-Myeon (KR); Jung Min Lee, Guri-Si (KR); Ju Yeon Jeong, Chuncheon-Si (KR); Hyung Woo Ju, Namyangju-Si (KR)

(73) Assignee: Industry Academic Cooperation Foundation, Hallym U niversity, Chuncheon-Si (JP)

(21) Appl. No.: 15/315,935

(22) PCT Filed: Jun. 10, 2016
(86) PCT No.: PCT/KR2016/006149

§ 371 Date: Dec. 2, 2016

(30) Foreign Application Priority Data
Aug. 19, 2015 (KR) 10-2015-0116762

(51) Int. Cl. A61K 49/00; C12N 5/00

(52) U.S. Cl. CPC A61K 49/0056; C12N 5/0068; C12N 2533/50

(57) ABSTRACT

There is provided a method of preparing a fluorescent silk fibroin solution, which includes a) obtaining scoured fluorescent silk fibroin by adding transgenic fluorescent silkworm cocoons to an aqueous solution including a scouring agent, heating the transgenic fluorescent silkworm cocoons at 40 to 60° C. for 8 to 24 hours and washing the transgenic fluorescent silkworm cocoons with distilled water, b) dissolving the scoured fluorescent silk fibroin at 40 to 60° C. for 1 to 5 hours in a solvent in which 15 mg to 1.5 g of dithiothreitol (DTT) is mixed per 100 mL of 9 to 9.6 M LiBr, and c) dialyzing the dissolved fluorescent silk fibroin in single distilled water for 48 to 96 hours. The scouring agent includes an alcalase and sodium hydrogen carbonate (NaHCO₃). The conventional methods of preparing a silk fibroin solution from fluorescent silkworm cocoons have drawbacks in that silk fibroin is not dissolved at a low temperature and fluorescence is lost. However, the method of the present invention, which includes adding a reducing agent, has an advantage in that a fluorescent silk fibroin solution can be prepared at low temperature, and fluorescence can be maintained. Therefore, since the fluorescent silk fibroin can be mass-produced, and a biocompatible fluorescent protein can be easily prepared and provided, materials applicable to the biotechnology industry such as supports for tissue regeneration, biosensors using bioimaging and biochips, etc. can be provided.

### TECHNICAL FIELD

The present invention relates to a method of preparing a fluorescent silk protein solution to which a reducing agent is added, and more particularly, to a method of preparing a fluorescent silk protein solution capable of obtaining a large amount of a protein from a transgenic silkworm cocoon using a reducing agent while maintaining fluorescence, and the manufacture and use of a support using the same.

### BACKGROUND ART

Silk fibroin is a typical natural polymer material manufactured by extracting silk from silkworm cocoons, and thus has been used as a fibrous material for textile, and a biomedical material such as suture thread for a long time. Silk fibroin has an excellent cell adhesion activity and proliferation effect on fibroblasts or keratinocytes without causing inflammatory reactions when applied to living bodies, and thus has received much attention as a material for artificial skin exhibiting excellent biocompatibility. Also, unlike other natural polymer material, silk fibroin may be easily obtained in large quantities as a pure protein from insects, has excellent biocompatibility, and thus is characterized by causing no rejection reactions in human bodies and being formed into various shapes such as a powder, a membrane, a porous material, a gel, etc. without undergoing a certain purification process.

In 1962, a green fluorescent protein (GFP) was first found by a marine biologist Shimomura Osamu (Japan) who had conducted research on a fluorescent material from a jellyfish (Aequorea victoria), and then named green fluorescent protein by Hasting and Morin in 1969. The GFP serves as an energy transfer acceptor that carries energy of a photoprotein or a luciferase-oxyluciferin complex that is activated in vivo by calcium ions, and also serves as a secondary fluorescent protein that receives energy from aequorin and emits green fluorescence at 508 nm. When DNA or mRNA coding for the GFP is present in cells, a GFP protein is synthesized in the cells to emit strong fluorescence. When a sequence coding for the GFP is linked to a gene whose expression level needs to be examined, the GFP emits fluorescence at a place at which the desired gene to be examined is actually expressed. Therefore, the GFP has been widely used to examine gene expression. In particular, due to its advantages such as non-toxicity and ease in being observed, the GFP has been used in various areas including determining whether a gene is expressed, measurement of a gene expression site and time, etc. A yellow fluorescent protein (YFP) and a cyan fluorescent protein (CFP) functionally similar to the GFP were also developed. In addition, fluorescent proteins emitting various colors such as red, yellow, and the like have been prepared. A variety of such fluorescent proteins are effectively used when a local distribution of various proteins is determined at one time.

A transgenic silkworm is prepared by inserting a green fluorescent gene from Aequorea victoria (North America) into a gene of fibroin (a fiber protein constituting a fiber from a silkworm cocoon) as a main component of a silk thread (a thread extracted from the silkworm cocoon), and then injecting the fibroin gene into an egg of a silkworm using a microinjector. This silkworm is a commercial silkworm race which is very difficult to transform, and may produce green fluorescent silk fibers three times or more than a transgenic silkworm prepared from a multivoltine silkworm race (having a nature of hatching eggs several times a year) in Japan. In particular, with the acquisition of unique source technology of finding the optimum microinjection site when a gene is injected into an egg of a silkworm, the transformation efficiency of the silkworm is improved up to 75% (42.5% on average), the value of which is far higher than that of Japan whose transformation efficiency is approximately 10%. The green fluorescent silk fibers extracted from the transgenic silkworm show bright green fluorescence in the dark when irradiated with light with certain wavelengths, and has a light green color when irradiated with natural light. Also, unlike color cocoons or golden cocoons whose color disappears in a scouring process of producing silk fibers, the cocoons of the transgenic silkworm maintain green fluorescence constant even when the cocoons are scoured. The green fluorescent gene is inherited by next-generation silkworms. Therefore, the green fluorescent silk fibers may be used as materials for fashion styles, wallpaper, lighting hats, accessories, interior supplies, etc. without an additional dyeing process.

A solution for manufacturing a fluorescent silk fibroin support cannot maintain fluorescence using conventional manufacturing methods. The green fluorescent protein (GFP) loses fluorescence as the GFP reacts with a strong base. Therefore, conventional silk fibroin solutions cannot maintain the fluorescence of the GFP since silk fibroin fibers obtained by scouring in the manufacturing method are dissolved in a strongly alkaline 9.5 M LiBr aqueous solution while the silk fibroin fibers are prepared into a solution. Also, unlike the conventional B. mori silk fibroin, the silk fibroin fibers are not easily dissolved in a 9.5 M LiBr aqueous solution, and thus have limitations in preparing a solution of the silk fibroin fibers.

### DISCLOSURE

### Technical Problem

To solve the above problems, the present inventors have conducted research to prepare a solution using fluorescent silkworm cocoons while maintaining fluorescence, and manufacture a support using the solution, and have found a method of preparing a solution to which a reducing agent is added.

### Technical Solution

According to an aspect of the present invention, there is provided a method of preparing a fluorescent silk fibroin solution, which includes a) obtaining scoured fluorescent silk fibroin by adding transgenic fluorescent silkworm cocoons to an aqueous solution including a scouring agent, heating the transgenic fluorescent silkworm cocoons at 40 to 60° C. for 8 to 24 hours and washing the transgenic fluorescent silkworm cocoons with distilled water, b) dissolving the scoured fluorescent silk fibroin at 40 to 60° C. for 1 to 5 hours in a solvent in which 15 mg to 1.5 g of dithiothreitol (DTT) is mixed per 100 mL of 9 to 9.6 M LiBr, and c) dialyzing the dissolved fluorescent silk fibroin in single distilled water for 48 to 96 hours. Here, the scouring agent may include an alcalase and sodium hydrogen carbonate (NaHCO₃).

According to another aspect of the present invention, there is provided a support for tissue regeneration, which includes the fluorescent silk fibroin solution prepared by the preparation method.

According to still another aspect of the present invention, there is provided a composition for bioimaging, which includes the fluorescent silk fibroin solution prepared by the preparation method.

### Advantageous Effects

The conventional methods of preparing a silk fibroin solution from fluorescent silkworm cocoons have drawbacks in that silk fibroin is not dissolved at low temperature and fluorescence is lost. However, the method of the present invention, which includes adding a reducing agent, has an advantage in that a fluorescent silk fibroin solution can be prepared at low temperature, and fluorescence can be maintained. Therefore, according to the present invention, since the fluorescent silk fibroin can be mass-produced, and a biocompatible fluorescent protein can be easily prepared and provided, materials applicable to the biotechnology industry such as supports for tissue regeneration, biosensors using bioimaging and biochips, etc. can be provided. Also, according to the present invention, a large amount of a fluorescent silk protein can be obtained from genetically modified silkworm cocoons, can be manufactured into various desired shapes, and can replace biocompatible biomaterials due to no cytotoxicity. Further, since the fluorescent silk protein did not lose the fluorescence even when manufactured into fluorescent silk materials, the fluorescent silk protein can contribute to biomedical fields requiring fluorescence, as well as the medical field.

### DESCRIPTION OF DRAWINGS

FIG. 1 shows a method of preparing a fluorescent silk fibroin solution.

FIG. 2 shows results of determining a molecular weight of fluorescent silk fibroin using sodium dodecyl sulfate-polyacrylamide gel electrophoresis (SDS-PAGE).

FIG. 3 shows results of determining the fluorescence of fluorescent silk fibroin solutions.

FIG. 4 shows fluorescence and cell proliferation results of observing fluorescent silk fibroin membranes using a confocal microscope.

FIG. 5 shows results of cytotoxicity tests on cells cultured on fluorescent silk fibroin membranes.

FIG. 6 is a diagram showing fluorescent silk fibroin nanomatrix observed under a scanning electron microscope (SEM) and a confocal microscope.

FIG. 7 is an image of animals observed after a fluorescent silk fibroin sponge is implated into the animals.

FIG. 8 is an image of tissues from the animals into which the fluorescent silk fibroin sponge is implated.

FIG. 9 is an image obtained by tracking an esophageal perforation in a mouse using fluorescent silk fibroin.

FIG. 10 is an image of tissues with respect to a fluorescent protein absorbed into the stomach (A: control, and B: EGFP-SF).

FIG. 11 is an image of p53 expression observed in cancer cells using a fluorescent SF-labeled p53 antibody.

### BEST MODE

The present invention provides a method of manufacturing a biocompatible fluorescent protein and an applied support to which a reducing agent is added to maintain fluorescence of fluorescent silkworm cocoons. According to the present invention, a large amount of a fluorescent silk fibroin protein may be produced from a small amount of transgenic fluorescent silkworm cocoons.

The method of preparing a fluorescent silk fibroin solution according to the present invention is characterized by including a) obtaining scoured fluorescent silk fibroin by adding fluorescent silkworm cocoons to an aqueous solution including a scouring agent, heating the fluorescent silkworm cocoons at 40 to 60° C. for 8 to 24 hours and washing the fluorescent silkworm cocoons with distilled water; b) dissolving the scoured fluorescent silk fibroin at 40 to 60° C. for 1 to 5 hours in a solvent in which 15 mg to 1.5 g of dithiothreitol (DTT) is mixed per 100 mL of 9 to 9.6 M LiBr; and c) dialyzing the dissolved fluorescent silk fibroin in single distilled water for 48 to 96 hours. The scouring agent preferably includes an alcalase and sodium hydrogen carbonate (NaHCO₃). In step a), a sericin protein may not be completely removed when the scouring time is less than 8 hours, whereas the fibroin protein may be denatured when the scouring time is greater than 24 hours. Also, in step a), an enzyme scouring agent such as an alcalase and the like may not be activated when the temperature is less than 40° C., whereas a fluorescent protein may be denatured when the temperature is greater than 60° C. In step b), the fluorescent silk fibroin protein may not be dissolved when the concentration of LiBr is less than 9 M, whereas LiBr may not be dissolved at room temperature when the concentration of LiBr is greater than 9.6 M. Also, in step b), fluorescence may not be expressed when the concentration of DTT is less than 15 mg, whereas the toxicity may be expressed when the concentration of DTT is greater than 1.5 g. Therefore, the fluorescent silk fibroin may not be suitable for use as a biomaterial. In addition, in step b), the fluorescent silk fibroin may not be dissolved when the temperature is less than 40° C., whereas the fluorescent protein may be denatured when the temperature is greater than 60° C. Also, the fluorescent silk fibroin may not be completely dissolved when the dissolution time is less than an hour, whereas a decrease in molecular weight of the fluorescent silk fibroin may be caused when the dissolution time is greater than 5 hours. In step c), a salt may not be removed from the fluorescent silk fibroin solution when the dialysis time in the fluorescent silk fibroin solution is less than 48 hours, whereas the silk fibroin protein may be denatured when the dialysis time is greater than 96 hours.

Hereinafter, the present invention will be described with reference of examples thereof. However, it should be understood that the following examples are just preferred examples for the purpose of illustration only and are not intended to limit or define the scope of the invention.

### EXAMPLE 1

1. Preparation of Fluorescent Silk Fibroin Solution

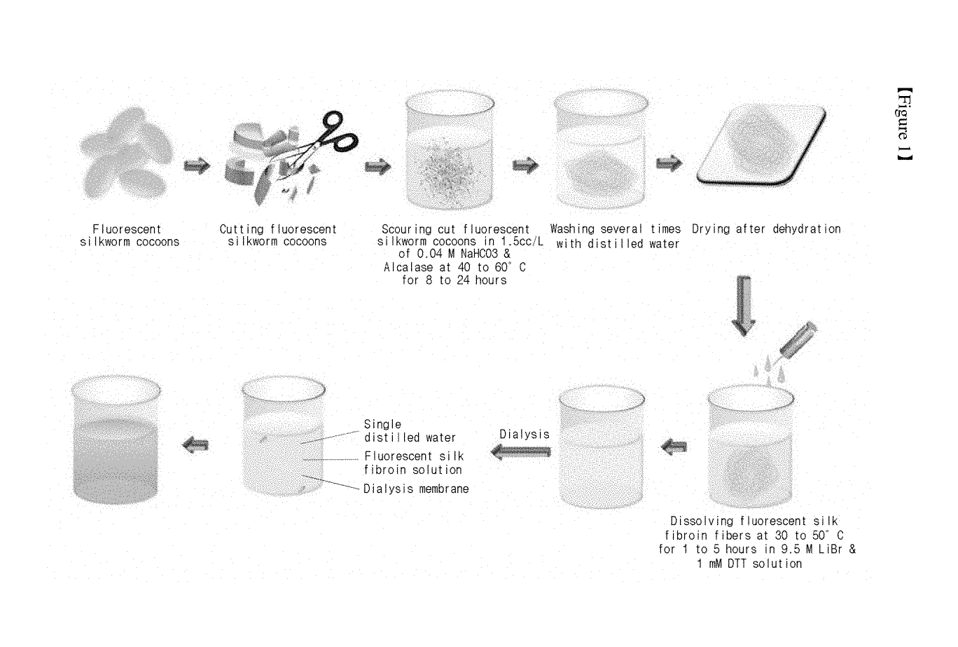

A scouring process, which includes adding 20 g of fluorescent silkworm cocoons to scouring water prepared by adding 0.9 mL of an alcalase and 1.8 g of NaHCO₃ to 600 mL of distilled water, heating the fluorescent silkworm cocoons at 60° C. for 12 hours, repeatedly washing the fluorescent silkworm cocoons three times with distilled water, was performed, as shown in FIG. 1. In this way, a sericin protein and impurities were removed to obtain 15 g of pure fluorescent silk fibroin fibers. The scoured fluorescent silk fibroin was dissolved at 40° C. for 3 hours in 100 mL of 9.5 M LiBr mixed with 1 mM dithiothreitol (DTT; 1 mM DTT was prepared by adding 15.41 mg of DTT, and fluorescence was expressed when a concentration of DTT was greater than 1 mM), and then dialyzed in single distilled water for 72 hours to prepare 200 mL of a pure fluorescent silk fibroin solution (4 w/w %) (FIG. 1).

2. Determination of Molecular Weight of Fluorescent Silk Fibroin

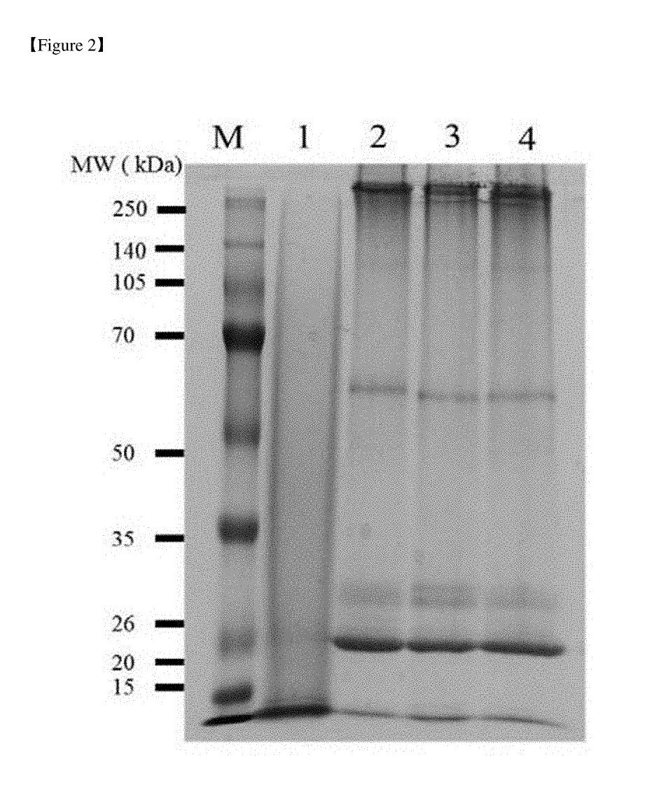

To determine a molecular weight of the fluorescent silk fibroin thus produced, SDS-PAGE was performed. A Mini-PROTEAN 3 Cell system (Bio-Rad) was used for SDS-PAGE. As test samples, four silk fibroin solutions having different colors (B. mori silk fibroin, EGFP, mKate2, and EYFP) were prepared, and run on 10% acrylamide gel and 4% condensing gel. Protein bands were stained with 0.25% Coomassie Brilliant Blue R-250 (Sigma-Aldrich, St. Louis, Mo., USA) to determine a molecular weight of the fluorescent silk fibroin (FIG. 2). It can be seen that B. mori silk fibroin (lane 1) was smeared in a range of 15 to 250 kDa. EGFP (lane 2), mKate2 (lane 3), and EYFP (lane 4) were observed to have approximately 60 kDa heavy chains and also 26 kDa light chains.

3. Determination of Fluorescence of Fluorescent Silk Fibroin Solution

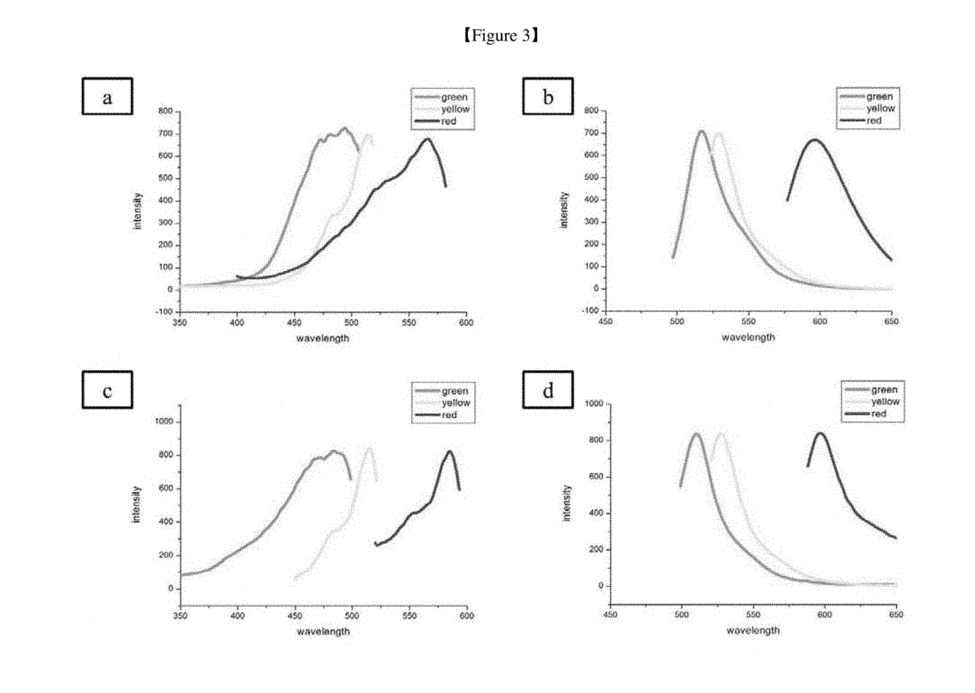

The fluorescence spectrum of the prepared 4% fluorescent silk fibroin solution was observed using an LS-55 fluorescence spectrophotometer (Perkin Elmer, Santa Clara, Calif., USA). It was known that the fluorescent proteins had excitation wavelengths at 488 nm (EGFP), 514 nm (EYFP), and 588 nm (mKate2) and emission wavelengths at 507 nm (EGFP), 527 nm (EYFP), and 633 nm (mKate2). As a result, it was confirmed that the excitation wavelengths of the fluorescent silkworm cocoons (FIG. 3A) and the fluorescent silk fibroin solution (FIG. 3C) were similar to each other, and also similar to the wavelengths of the conventional fluorescent proteins. Also, it was confirmed that the emission wavelengths of the fluorescent silkworm cocoons (FIG. 3B) and the fluorescent silk fibroin solution (FIG. 3D) were similar to each other, and also similar to the emission wavelengths of the conventional fluorescent proteins (FIG. 3). Therefore, it was revealed that the fluorescent silk fibroin solution prepared from the fluorescent silkworm cocoons maintained fluorescence.

### EXAMPLE 2

Manufacture of Fluorescent Silk Fibroin Membrane and Determination of Cell Proliferation

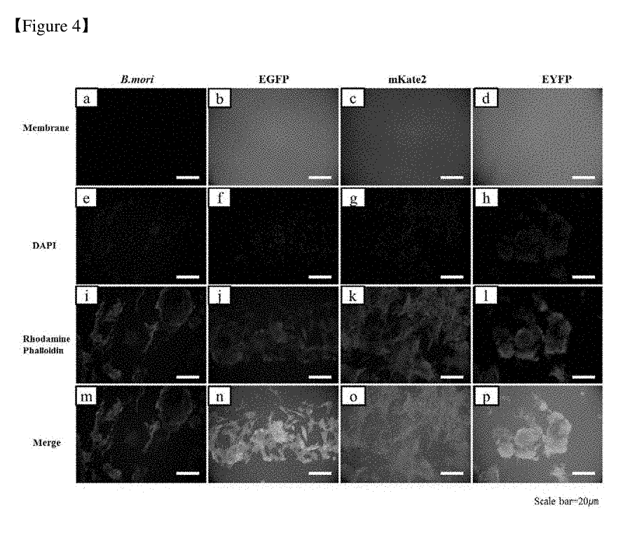

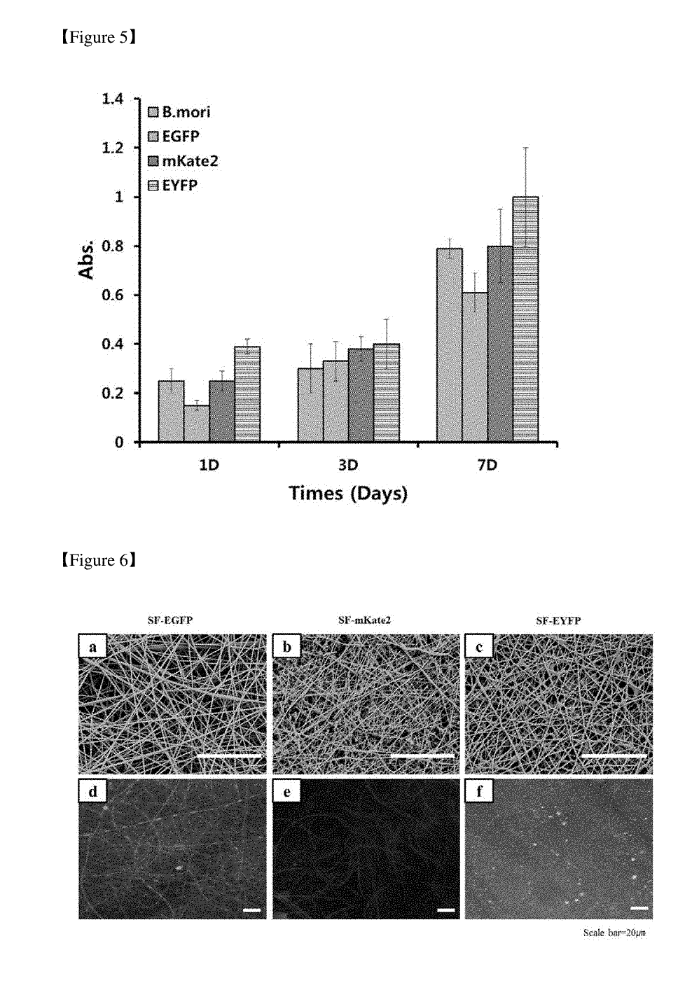

A fluorescent silk fibroin membrane was manufactured, as follows. 5 to 10 mL of the prepared fluorescent silk fibroin solution was poured into a 90×15 mm Petri dish, and dried indoors at an average temperature of 26° C. for 48 hours. Each of the fluorescent silk fibroin films formed by drying was made into circles using a biopsy punch having a diameter of 3 mm, and NIH 3T3 fibroblasts were seeded in a 96-well cell culture plate, and then cultured for 24 hours. For observation with the naked eye, the cells were stained with 4′,6-diamidino-2-phenylindole (DAPI) and rhodamine phalloidin (Invitrogen Co., San Diego, Calif., USA), and observed under a fluorescence microscope (Eclipse 80i, Nikon Co., Japan). As a result, it was observed that the fluorescent silk fibroin film maintained its innate fluorescence, and also had excellent cell adhesion (FIG. 4). Also, to check a cell culture level and cell affinity of the fluorescent silk fibroin film, a CCK-8 assay was carried out after 1, 3, and 7 days of culturing. As a result, it was revealed that the number of the cells seeded onto each of the supports tended to gradually increase with time (FIG. 5).

Therefore, it was revealed that the fluorescent silk fibroin according to the present invention maintained innate fluorescence without losing fluorescence even when manufactured into films, and that the fluorescent silk fibroin was easily attached to the support during the cell culture.

### EXAMPLE 3

Manufacture and Determination of Fluorescent Silk Fibroin Nanomatrix

A fluorescent fibroin nanofiber matrix was manufactured, as follows. The fluorescent silk fibroin sponge manufactured in Example 2 was dissolved at 40° C. for 16 hours in 6.5% (w/v) of a solvent serving as a 98% formic acid aqueous solution. The prepared solution was put into a 10-mL syringe. The solution was sprayed through four syringes for 12 hours. In this case, the flow rate was maintained at 0.3 mL/h, a 22-gauge syringe tip (0.7 mm OD×0.4 mm ID) was used, the distance between the syringe tip and a collector (tip-to-collector distance (TCD)) was set to 15 cm, and the syringe tip and the collector were set to have a voltage of +20 kV and −2kV, respectively.

To determine whether nanofibers and fibers of the manufactured fluorescent silk fibroin nanomatrix maintain fluorescence, the nanofibers and fibers were observed using a scanning electron microscope (SEM) and a confocal microscope. Based on the SEM images (FIGS. 6A, 6B and 6C), it was revealed that the nano-fibers were able to be manufactured using electrospinning. Based on the confocal microscope images (FIG. 6D, 6E and 6F), it was revealed that the nano-fibers maintained fluorescence (FIG. 6).

### EXAMPLE 4

In vivo Fluorescent Image Using Animal Model

1. Biophotonics Through In vivo Insertion of Silk Sponge

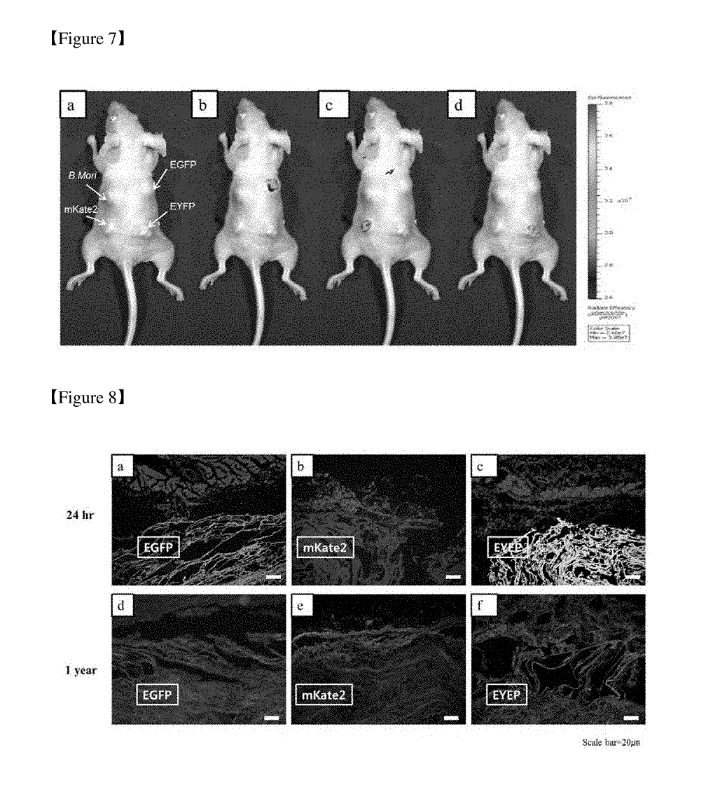

The prepared fluorescent silk fibroin solution was frozen at −80° C. for 12 hours, and then lyophilized for 24 to 36 hours to manufacture a fluorescent silk fibroin sponge. The manufactured fluorescent silk fibroin sponge was inserted into an animal model, and an in vivo fluorescent image effect was confirmed. Hairless mice were used as the animal model, and it was confirmed that the mice emitted fluorescence in vivo using a fluorescence animal imaging system (IVIS 200, Perkin Elmer, Santa Clara, Calif., USA). From the confirmation results, it was observed that the fluorescence was emitted from tissues containing the sponge (FIG. 7). Also, tissues were extracted within 24 hours and after one year, and subjected to cryosection to prepare samples. For observation with the naked eye, the cells were then stained with 4′,6-diamidino-2-phenylindole (DAPI) and rhodamine phalloidin (Invitrogen Co., San Diego, Calif., USA), and observed using a fluorescence microscope (Eclipse 80i, Nikon Co., Japan) (FIG. 8). As a result, it was observed that all the supports in the samples extracted within 24 hours (FIGS. 8A, 8B and 8C) and the samples extracted after one year (FIGS. 8D, 8E and 8F) maintained their shape and fluorescence.

2. Esophageal Perforation Model

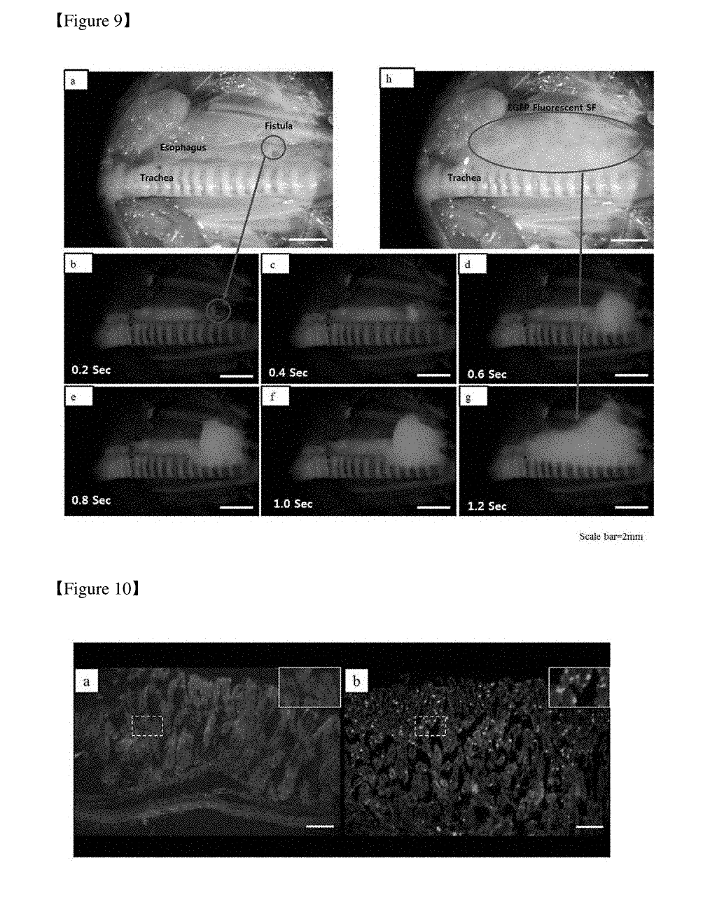

In this study, the prepared fluorescent silk fibroin solution was applied to an animal model to determine whether the fluorescent silk fibroin solution was applicable to biophotonics. The esophagi of SD rats used in the animal model were perforated at a diameter of 0.5 mm, and the prepared fluorescent silk fibroin solution was orally administered to the SD rats. Thereafter, the SD rats were observed every two seconds using a fluorescence microscope. Based on the observation results, it was revealed that the fluorescent material was detected at the perforation sites over time, and the minute perforation sites were tracked (FIG. 9).

3. Histological Observation of Fluorescent Protein Absorbed into the Stomach

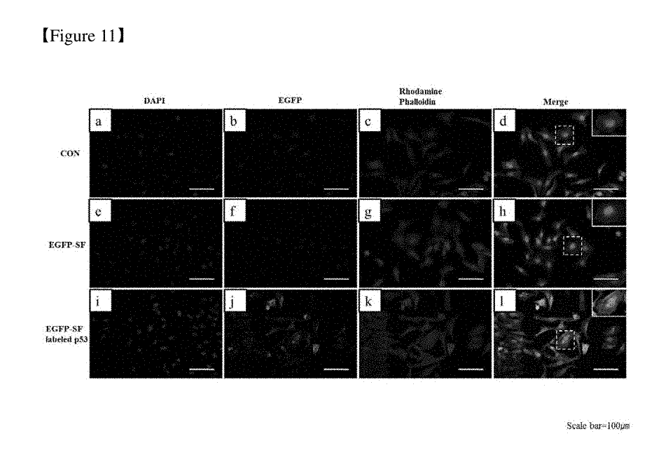

1 g of the fluorescent silk fibroin sponge manufactured in Example 2 was orally administered to SD rats to track a fluorescent material absorbed into the stomach. Tissues were extracted from the stomach after 24 hours of oral administration of the fluorescent silk fibroin sponge. As the control, tissues were extracted after 1 g of a B. Mori silk sponge was orally administered to SD rats. The extracted tissues were subjected to cryosection to prepare samples. For visual observation, the cells were then stained with 4′,6-diamidino-2-phenylindole (DAPI), and observed using a fluorescence microscope (Eclipse 80i, Nikon Co., Japan). As a result, it was revealed that the fluorescent particles were not observed when the B. Mori silk sponge was orally administered as the control (FIG. 10A), but the fluorescent particles were distributed in the gastric mucosa when the fluorescent silk fibroin sponge of the present invention was orally administered (FIG. 10B) (FIG. 10). Therefore, it was confirmed that the fluorescent silk fibroin particles did not lose fluorescence when exposed to gastric acid, and thus was applicable as a biophotonic biomaterial capable of permeating into the gastric mucosa.

4. Manufacture of Fluorescent SF-Labeled P53 Antibody and Determination of P53 Expression in Cancer Cells using the Same

154.05 μL of an NHS-PEG4-biotin solution (an EZ-link NHS-PEG4-biotinylation kit, Thermo Fisher Scientific, Waltham, Mass., USA) was added to 1 mL of a purified fluorescent silk solution diluted to 0.2%, and incubated on ice for 4 hours, and the resulting mixture was then allowed to pass through a desalting column to prepare a biotinylated silk solution. 7 μL of a p53 antibody (antibody concentration: 1 mg/mL; Abcore Inc, Ramona, Calif., USA) was mixed in 500 μL of the solution, incubated for 2 hours to manufacture a fluorescent SF-labeled p53 antibody. HeLa cells were cultured in a 96-well plate, and treated with the manufactured antibody at a dose of 300 μL per well. After 24 hours, the HeLa cells were observed using a fluorescence microscope (Eclipse 80i, Nikon Co., Japan) (FIG. 11). It could be seen that a p53 gene was overexpressed in the HeLa cells using the fluorescence-tagged antibody.

1. A method of preparing a fluorescent silk fibroin solution, comprising:
a) obtaining scoured fluorescent silk fibroin by adding transgenic fluorescent silkworm cocoons to an aqueous solution including a scouring agent, heating the transgenic fluorescent silkworm cocoons at 40 to 60° C. for 8 to 24 hours and washing the transgenic fluorescent silkworm cocoons with distilled water;
b) dissolving the scoured fluorescent silk fibroin at 40 to 60° C. for 1 to 5 hours in a solvent in which 15 mg to 1.5 g of dithiothreitol (DTT) is mixed per 100 mL of 9 to 9.6 M LiBr, and
c) dialyzing the dissolved fluorescent silk fibroin in single distilled water for 48 to 96 hours.

2. The method of claim 1, wherein the scouring agent comprises an alcalase and sodium hydrogen carbonate (NaHCO₃).

3. A support for tissue regeneration comprising the fluorescent silk fibroin solution prepared from the transgenic fluorescent silkworm cocoon according to the preparation method defined in claim 1.

4. A composition for bioimaging comprising the fluorescent silk fibroin solution prepared from the transgenic fluorescent silkworm cocoon according to the preparation method defined in claim 1.

5. A support for tissue regeneration comprising the fluorescent silk fibroin. solution prepared from the transgenic fluorescent silkworm cocoon according to the preparation method defined in claim 2.

6. A composition for bioimaging comprising the fluorescent silk fibroin solution prepared from the transgenic fluorescent silkworm cocoon according to the preparation method defined in claim 2.
